(19) United States
(12) Patent Application Publication
(10) Pub. No.: US 2023/0324360 A1
(43) Pub. Date: Oct. 12, 2023

(54) INDUSTRIAL WATER ANALYSIS DEVICE AND SUPPORT THEREFOR

(71) Applicant: HORIBA TOCADERO GMBH, Berlin (DE)

(72) Inventors: Oliver ROTHE, Steinhagen (DE); Rafael JECHOREK, Berlin (DE); Mike SCHONFELDER, Berlin (DE); Frank BEHRENDT, Berlin (DE)

(21) Appl. No.: 18/043,107

(22) PCT Filed: Aug. 28, 2020
(86) PCT No.: PCT/EP2020/074142

§ 371 Date: Feb. 27, 2023

(51) Int. Cl. G01N 33/18; G01N 31/12

(52) U.S. Cl. CPC G01N 33/1846; G01N 31/12

(57) ABSTRACT

The invention relates to an industrial water analysis device (1), in particular to an industrial TOC and/or TNb water analysis. The industrial water analysis device comprises a housing (2) with an access opening (60). The industrial water analysis device further comprises a water analysis assembly (6) that is located within the housing and comprises at least one of a heating assembly (12) and a reactor assembly (10). The reactor assembly may comprise a reactor tube (14). In order to facilitate maintenance, a mounting assembly (70) is provided according to the invention, which is attached to the housing (2) and to the water analysis assembly (6). The water analysis assembly (6) is moveable relative to the housing from an operating position (56) to a maintenance position (80), while remaining attached to the mounting assembly. In the maintenance position, at least parts of the water analysis assembly (6) are moved out of the operating position in a direction (81) pointing towards the access opening (60). In particular, part of the water analysis assembly may protrude out of the housing in the maintenance position.

The invention relates to an industrial water analysis device, in particular an industrial TOC (Total Organic Carbon) or TNb (Total Nitrogen bound) analysis.

An industrial water analysis device is used e.g. for monitoring the water quality of water that is used in industrial processes or in wastewater. The sum parameters TOC and TNb are among the most important parameters in the water and wastewater analysis.

Industrial water analysis devices are inline devices, which are configured to automatically take and analyze water probes around the clock. Any downtime of an industrial water analysis device may create huge costs as entire facilities and processes must be stopped. Therefore, there is a need that industrial water analysis devices are maintained and repaired quickly and efficiently with minimum downtime.

This need is addressed according to the invention by an industrial water analysis device, in particular an industrial TOC and/or TNb water analysis device, comprising: a housing, the housing having an access opening; a water analysis assembly, the water analysis assembly being arranged within the housing and comprising at least one of a heating assembly and a reactor assembly, the water analysis assembly further comprising a top section and a bottom section, the top section being located above the bottom section in a vertical direction; and a mounting assembly, the mounting assembly being attached to the housing; wherein the water analysis assembly is attached to the mounting assembly while being moveable relative to the housing from an operating position to a maintenance position; wherein, in the maintenance position, at least parts of the water analysis assembly are moved out of the operating position in a direction pointing towards the access opening.

By being moved towards the access opening in the maintenance position, access to the water analysis assembly and its components is facilitated. There is more room available to carry out all necessary work, which therefore can be performed faster and more efficiently.

The invention can be further improved by any of the following advantageous features, which can be combined arbitrarily and independently of one another.

For example, the water analysis assembly may, in the maintenance position, at least partly protrude out of the housing. By protruding at least partly out of the housing, access to the water analysis assembly and its constituents is further facilitated. The space for doing maintenance work is no more limited to the interior of the housing.

According to another advantageous embodiment, at least the top section of the water analysis assembly is moved out of the housing in the maintenance position. This facilitates access to the top section.

In the maintenance position, the water analysis assembly may be rotated relative to the operating position. As the water analysis assembly is often connected via inflow fluid lines and outflow fluid lines to other components, a rotation allows maintaining at least those connections in the maintenance position that are located close to the axis of rotation. Further, the mechanical expense for a pure rotational movement is small. For effecting a rotational movement between the maintenance position and the operating position, a joint, in particular a hinge may be provided.

Of course, instead of being rotated, the water analysis assembly may, alternatively or additionally, be moved translationally from the operating position to the maintenance position and/or back.

A translational movement may be realized in that the mounting assembly comprises a telescopic pullout and/or a drawer. For example, the water analysis assembly may rest and be fastened to such a drawer, or to a plate resting on a telescopic pullout.

It is preferred that the joint is located closer to the bottom section than to the top section of the water analysis assembly. In this arrangement, the location of the axis of rotation, which may be defined by the joint, is easily possible to rotate the top section of the water analysis assembly out of the housing. In particular, the joint may be located at or even below the bottom section of the water analysis assembly, so that in the maintenance position no part of the water analysis assembly is rotated away from the access opening.

The joint may be located, according to another embodiment, closer to a front side of the water analysis assembly than to a backside of the water analysis assembly. The front face of the water analysis assembly hereby faces the access opening, whereas the rear side faces away from the access opening. The further the joint is located towards the front side of the water analysis assembly, the less the water analysis assembly will rotate below its position in the operating position. In particular, if the joint is located at the front side of the water analysis assembly or between the front side and the access opening, the entire water analysis assembly will rotate in an upward direction from the operating position to the maintenance position, i.e. away from any support on which the water analysis assembly rests in the operating position.

In order to provide further mechanical support during the movement between the maintenance position and the operating position, a guide system may be provided. The guide system may be comprised by the mounting assembly, e.g. be unitarily integrated into the mounting assembly.

The guide system may further provide a limit stop which restricts movement out of the operating position and defines the maintenance position.

The guide system may be spaced apart from the joint and comprise a guide slot or rail along which one of the water analysis assembly and the mounting assembly is guided relative to the other one of the water analysis assembly and the mounting assembly during the movement between the maintenance position and the operating position.

The mounting assembly may comprise a moveable part and a stationary part. The moveable part may be fastened to the water analysis assembly and the stationary part may be fastened directly or indirectly to the housing. Spacers may be provided between the moveable part and the water analysis assembly and/or between the stationary part and the housing. The moveable part and the stationary part may be connected to one another by the joint.

If a guide system is used, part of the guide system may be located on the moveable part and another part of the guide system may be part of the stationary part. The moveable part may comprise, for example, a sliding member, which slidingly engages the slot or rail that may be part of the stationary part. Integrating the guide system in the mounting assembly facilitates the assembly of the industrial water analysis device. Further, it stabilizes mechanically the mounting assembly.

The mounting assembly may further comprise a frame into which the water analysis assembly is inserted. The frame may be basket-like or cage-like. For example, the frame may comprise a plurality of stanchions which, in the operating position, extend along side faces of the industrial water analysis device. For example, at least one stanchion may rest against a side face of the water analysis assembly, securing the water analysis assembly in a direction parallel to the access opening. Alternatively or additionally, at least one stanchion may be located at the front side of the water assembly. This stanchion may add further support, if the water analysis assembly is rotated out of the operating position into the maintenance position. At least one stanchion may have an L-shaped cross section and extend along an edge of the water analysis assembly, abutting the two faces of the water analysis assembly that meet at this edge.

The frame, into which the water analysis assembly is inserted may open horizontally towards the access opening. This allows insertion of the water analysis assembly into the mounting assembly through the access opening. In another embodiment, the frame may open vertically. In this configuration, the water analysis assembly may be inserted into the frame from above in the maintenance position.

The water analysis assembly may comprise a housing, which is termed interior housing in the following, as it is located inside the housing, which may also be termed outer housing, providing the access opening. In the interior housing, at least the reactor assembly, preferably also the heating assembly may be received. The interior housing may comprise a heat insulation, which is particularly advantageous if the water analysis assembly comprises a heating assembly.

The heating assembly may be configured to heat up the reactor assembly at least in parts up to 1200° C. At this temperature, carbon is oxidized for TOC analysis.

The interior housing may have a circular or polygonal, in particular rectangular base area. It may be fabricated from sheet material, such as metal sheets.

The interior housing may comprise a top maintenance opening at the top section and/or a bottom maintenance opening at the bottom section. Any of these maintenance openings allows access to the interior of the interior housing and thus to the components of the water analysis assembly. Part of the water analysis assembly may protrude out of at least one of the top and the bottom maintenance opening. The bottom maintenance opening may be aligned with and/or overlap the at least one opening of the mounting assembly, if present.

According to another advantageous embodiment, the reactor assembly protrudes out of the interior housing through the top maintenance opening at the top section. This allows easy access to the reactor assembly if it is moved closer to or out of the access opening in the maintenance position.

The reactor assembly may comprise a reactor tube, which protrudes through the maintenance opening. The reactor tube provides the reactor volume, in which oxidation of the carbon and evaporation of the water takes place. The reactor tube may be arranged preferably coaxially within the reactor assembly. In the operating position, a longitudinal axis of the reactor tube is preferably arranged vertically.

Attachment means may be provided, which are preferably located in the top section and preferably outside the interior housing. The attachment means fasten at least one of the reactor assembly and the reactor tube to the remainder of the water analysis assembly, in particular the interior housing. Preferably, the attachment means provide the only attachment points which fasten the reactor assembly and/or the reactor tube to the interior housing.

The attachment means are preferably arranged and configured to be operated and accessible from outside the interior housing, in particular manually without tools. Thus, they can be easily operated once the water analysis assembly has been moved into the maintenance position.

Releasing the attachment means in this configuration allows to detach the reactor assembly and/or the reactor tube from the interior housing or the remainder of the water analysis assembly.

If at least one of the reactor assembly and the reactor tube are configured to be removed from the interior housing or the remainder of the water analysis assembly through the maintenance opening, they can be easily and quickly exchanged without removing the entire water analysis assembly out of the internal housing and without unmounting the entire water analysis assembly from the housing that provides the access opening.

In another advantageous embodiment, the water analysis assembly is configured to be operable in the maintenance position. This is the case for example when the water analysis assembly comprises at least one inflow fluid line and at least one outflow fluid line and at least one electric power line that stay connected when the water analysis assembly is moved from the operating position into the maintenance position and/or from the maintenance position to the operating position. The at least one inflow fluid line is configured to supply fluids, such as the water to be analyzed, to the water analysis device. The at least one fluid outflow line is configured to direct fluids, such as purging or cleaning fluid, out of the water analysis device. The electric power line may supply electric power to electric components of the water analysis assembly, such as the heating assembly or any sensors that may be part of the water analysis device, such as an NDIR detector for detecting CO₂ an ICD detector for TNb and/or a CLD/NDUV detector also for detecting TNb.

The industrial water analysis device is preferably configured as an inline analysis device i.e. to stay continuously connected to a water source. The industrial water analysis device may comprise a controller, such as an ASIC, a processor or, generally, any kind of integrated circuit that is configured to operate the industrial water analysis device to automatically draw samples from the water source depending on a stored, predetermined schedule. The predetermined schedule may be stored in a storage section, such as a memory of the industrial water analysis device.

The water analysis assembly may comprise a reactor cover, which seals the reactor assembly and, in particular, the reactor tube against the environment. The reactor cover may comprise a connector for an inflow fluid line, in particular for supplying the water to be analyzed. The reactor cover may be fastened to the interior housing or the remainder of the water analysis assembly by the attachment means. The reactor assembly and/or the reactor tube may be attached to the reactor cover and removed from the remainder of the water analysis assembly, in particular the interior housing, together with the reactor cover. Alternatively, once the reactor cover is removed, the reactor assembly and/or the reactor tube are accessible and can be removed. For example, the reactor assembly and/or the reactor tube may rest against the interior housing and be suspended therefrom so that they can be pulled out of the top maintenance opening once the attachment means is released and the reactor cover is removed. The reactor cover may be located outside the interior housing and e.g. also cover the top maintenance opening.

The water analysis assembly may further comprise a bottom closure, which closes a bottom part of the reactor assembly and/or the reactor tube, preferably sealingly. The bottom closure may be fastened to the interior housing. The reactor assembly and/or the reactor tube may be inserted into the bottom closure and be in a sliding and preferably also sealing engagement with the bottom closure, to allow the reactor assembly and/or the reactor tube to be pulled out of the bottom closure through the top maintenance opening. The bottom closure may comprise one or more connectors for outflow fluid lines. The bottom closure may comprise a lower part, which forms a container, into which solid residuals from the oxidation process in the reactor assembly and/or the reactor tube may fall and be collected. The part of the bottom closure containing the container is preferably removeably without tools, e.g. by wing screws. The bottom closure may be arranged accessible through the access opening from outside the interior space of the housing. This may be accomplished in that a space between the bottom closure and the access opening is empty.

The mounting assembly, in particular if located below the water analysis assembly, may comprise at least one opening through which part of the water analysis assembly protrudes. For example, the bottom closure of the reactor assembly may protrude through the at least one opening.

The mounting assembly may be fixed to the housing in the operating position using a securing element, which is e.g. fastened to the housing. The one or more securing element is preferably configured to be operated manually without tools. For example, the at least one securing element may provide a handle for operating a thread or a bayonet, such as a wing screw. The mounting assembly may be attached to a rear wall of the housing and/or to a bottom of the housing. In the maintenance position the securing element may be detached or released.

The water analysis assembly is preferably assembled into a unit, which may be handled as a single piece. For example, the water analysis assembly may be separated from and mounted onto the mounting assembly as a pre-assembled unit.

According to another advantageous embodiment, the mounting assembly is configured to be mounted preferably as a preassembled unit in an existing industrial water analysis device. This embodiment allows to retrofit existing industrial water analysis devices with the mounting assembly and thus provide facilitated access via the maintenance position. The mounting assembly in this case may be configured in any form as described.

In the following, an embodiment of the invention is described exemplarily with reference to the drawings. In the drawings, elements that correspond to each other with respect to structure and/or function are indicated with the same reference numeral.

According to the various improvements to the invention as described above, one or more features can be omitted from the embodiment if, for a specific application, the technical effects associated with the one or more features are not needed. Likewise, one or more features described above, may be added to the embodiment if, for a specific application, the technical effect of this feature is needed or of advantage.

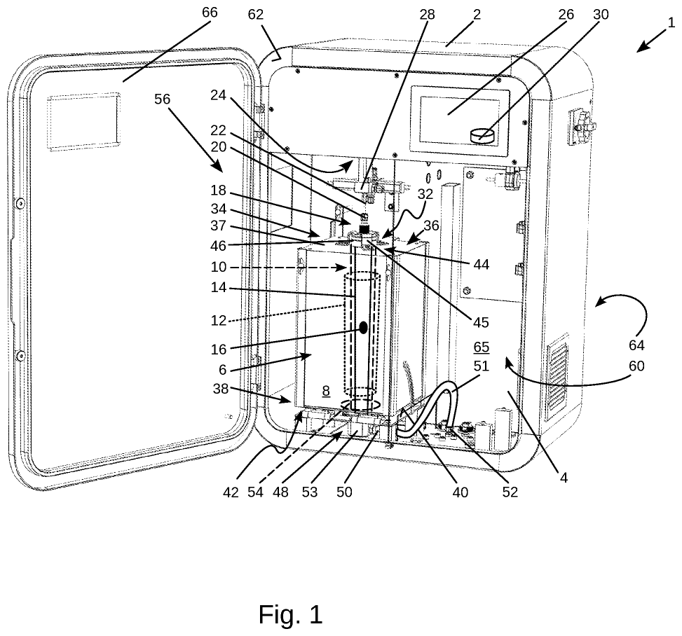

FIG. 1 shows a schematic, perspective front view of an industrial water analysis device in an operating position;

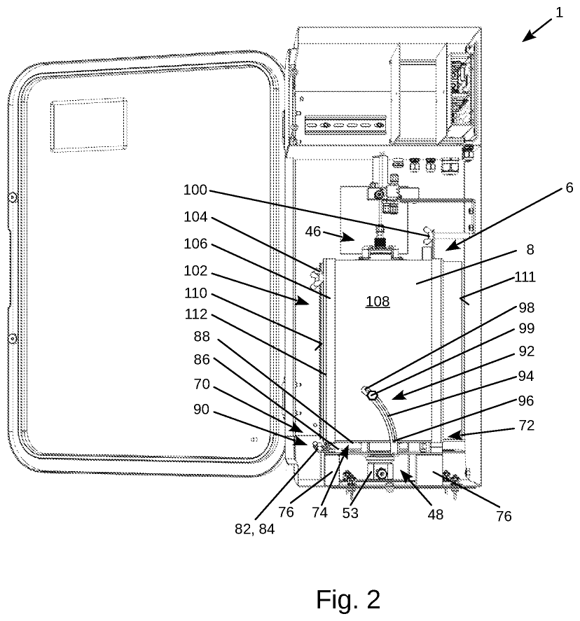

FIG. 2 shows a schematic, perspective side view of the water analysis device of FIG. 1 in the operating position;

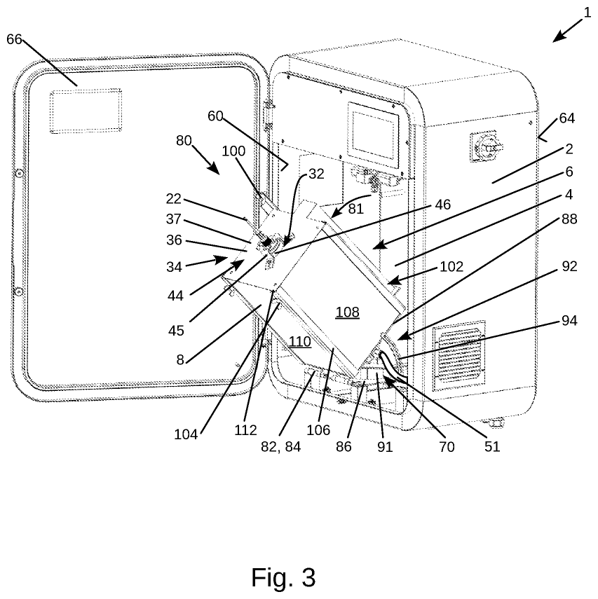

FIG. 3 shows a schematic, perspective side view of the industrial water analysis device of FIG. 1 in a maintenance position;

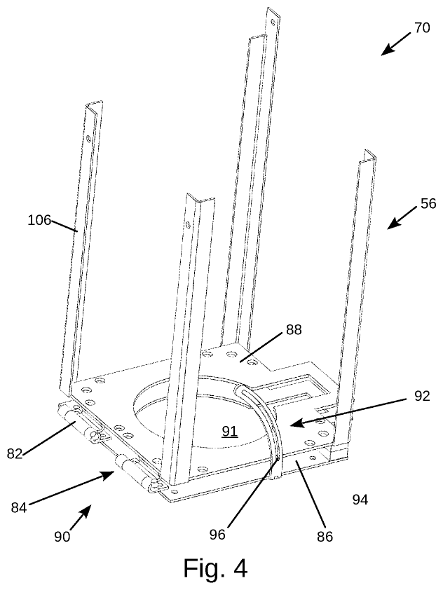

FIG. 4 shows a schematic, perspective view of a mounting assembly.

The structure and function of an industrial water analysis device 1 are explained with reference to FIGS. 1 to 4.

The industrial water analysis device 1 is configured for industrial use, i.e. for use 24/7 to continuously monitor specific contents in water, such as TOC (total organic carbon) or TNb (total nitrogen bound). In particular, the industrial water analysis device 1 may be configured to automatically draw and analyze water samples according to a predetermined schedule. This distinguishes the industrial water analysis device from a water analysis device for laboratory use, which is employed only sporadically, but which may have greater resolution and sensitivity for the components of the water to be identified.

The industrial water analysis device 1 comprises a housing 2 which encloses an interior space 4.

Within the housing 2, or in the interior space 4, a water analysis assembly 6 is arranged. The water analysis assembly 6 may comprise an interior housing 8, which is arranged within the outer housing 2. Within the housing 2 other components of the water analysis device may be arranged.

The water analysis assembly 6 is preferably assembled as a unit, i.e. all components of the water analysis assembly 6 may be mechanically integrated so that the water analysis assembly 6 can be inserted in and removed from the housing as a unit.

The water analysis assembly 6 preferably comprises all those components that are necessary for analyzing water, particularly for determining TOC and/or TNb. For example, the water analysis assembly 6 may comprise at least one of a reactor assembly 10 and a heating assembly 12.

The reactor assembly 10 is configured to receive and evaporate water. The heating assembly 12 is configured to heat the reactor assembly 10 to evaporate water and/or oxidation carbon. Specifically, the heating assembly 12 may be configured to heat the reactor assembly 10 at least section-wise to a temperature of at least 1000° C., preferably to about 1200° C. The heating assembly 12 may comprise an oven. The interior housing 8 may, on its inner side, be provided with a heat-insulation layer.

The reactor assembly 10 may comprise a reactor tube 14, which stands upright within the water analysis assembly 6 and receives water through a top opening. The reactor tube 14 is preferably made from a ceramic material and provides the actual chamber in which the water is heated and evaporated.

The water analysis assembly 6 may comprise a sensor assembly 16 which is configured to output data that are representative of the concentration of at least some components in the water that has been evaporated in the reactor assembly 10, particularly of the TOC and the TNb.

The sensor assembly 16 may have, for example, at least one of NDIR detector for detecting CO₂, an ECD detector for detecting Tb and a CLD/NDUV detector for detecting TNb. The measurement for TOC may be in conformity with DIN EN 1484 and of TNb in conformity with DIN EN 12260.

The water analysis assembly 6 may comprise a water inflow section 18, which may be provided with a standardized fluid connector 20 for connecting at least one inflow fluid line 22 e.g. from a water source 24 and/or one or more reservoirs of a chemical agent. The water inflow section 18 may be in fluid connection with the reactor assembly 10, particularly the reactor tube 14. In operation, the water to be analyzed by the industrial water analysis device 1 is fed from the water source through the water inflow section 18 to the water analysis assembly 6, particularly into the reactor assembly 10 or, specifically, the reactor tube 14.

The industrial water analysis device 1 may comprise a controller 26, such as an ASIC or a programmable integrated circuit that is configured to control the operation of industrial water analysis device 1. For example, a unidirectional or bidirectional data-transfer connection 27, such as a cable or a wireless connection may be established between the controller 26 and an inflow valve 28. The inflow valve may be arranged upstream of the water inflow section 18, e.g. in the fluid line 22 or at the water source 24.

The controller 26 is configured to open the inflow valve 28 depending on a predetermined schedule, which may be stored in a memory section 30 of the controller 26. The controller may be further configured to control the heating assembly 12 and any purging and cleaning operation of the water analysis assembly 6.

For example, the controller 26 may keep a temperature of the reactor assembly 10 or the reactor tube 14 at least in sections and at least approximately constant at or close to 1200° C. Upon opening the inflow valve 28, water enters the reactor assembly 10 and is evaporated. The controller 26 may be configured to initiate a TOC or TNb measurement using the sensor assembly 16 and to receive data representative of the TOC or TNb from the sensor assembly. The controller 26 is configured to purge the reactor assembly 10 once this data has been received from the sensor assembly 16. The controller may be configured to be connected to a computer network, e.g. by being configured to operate a WLAN or Ethernet connection.

The interior housing 8 may comprise a top maintenance opening 32 through which at least the reactor assembly 10 or the reactor tube 14 protrudes. The top maintenance opening 32 is located at a top section 34, in particular a top face 36 of the interior housing 8. The top face may be formed by a top plate 37, e.g. made from sheet metal. The top section 34 or the top face 36, respectively, are located vertically above a bottom section 38 of the interior housing 8. The bottom section 38 may comprise a bottom face 40, which may be formed by a bottom plate 42, which may be made from sheet metal.

The industrial water analysis device 1 may further comprise attachment means 44, such as clamps or brackets, which are configured to secure at least one of the reactor assembly 10 and the heating assembly 12, in particular the reactor tube 14, within the water analysis assembly 6. In particular, the attachment means may secure the reactor assembly 10, the heating assembly 12 and/or the reactor tube 14 to the interior housing 8. For this, any kind of fastening means, such as screws or bolts or clips may be used. The attachment means 44 are arranged on the top face 36 and are configured and located to be accessible from outside the water analysis assembly 6 and specifically from outside the interior housing 8, respectively.

The water analysis assembly 6, in particular the reactor assembly 10 may further comprise a reactor cover 46, which is adapted to seal the reactor assembly 10 and/or the reactor tube 14 to the outside and which comprises the water inflow section 18. The reactor cover further seals the maintenance opening 32 against leakage of hot air from the interior of the interior housing 8.

The reactor assembly 10, specifically the reactor tube 14 is fastened preferably solely by the attachment means 44 and/or the reactor cover 46 to the interior housing 8. Thus, by removing or releasing the attachment means 44, the reactor assembly 10, specifically the reactor tube 14 is configured to be removed through the maintenance opening 32 and the top section 34 from the interior housing 8. In such an embodiment no access to other parts of the interior housing 8, such as an axis to the bottom section 38 is required for the removal. Access to the top section 34 of the water analysis assembly 6 is sufficient.

The water analysis assembly 6 may further comprise a bottom closure 48, which may be located at the bottom section 38 of the water analysis assembly. In particular, the bottom closure 48 may constitute a bottom of the reactor assembly 10 or the reactor tube 14. The bottom closure 48 may be fastened to the bottom section 38, in particular the bottom plate 42, of the interior housing 8.

Preferably, the reactor assembly 10 and/or the reactor tube 14 is not fastened to the bottom closure 48, so that the reactor assembly 10 and/or the reactor tube 14 may be pulled from the bottom closure 48 once the attachment means 44 are released. The bottom closure may stay attached to the interior housing 8. For example, the reactor assembly 10 and/or the reactor tube 14 may be inserted into the bottom closure 48 and be held slidingly by a seal.

The bottom closure 48 may comprise one or more outflow connectors 50 through which fluid, such as water and/or cleaning fluid is directed out of the water analysis assembly 6. One or more outflow fluid lines 51 may be connected to the one or more outflow connectors 50 to ultimately drain the outflow fluids outside the housing 2. The outflow fluid lines may be connected to a drain 52 to direct the outflow fluids out of the industrial water analysis device 1. The bottom closure 48 may further comprise a removable container 53, in which solid residuals and other material may fall from the reactor assembly 10 and/or the reactor tube 14.

As can be seen in FIGS. 1 to 3, the bottom closure 48 preferably protrudes outside the interior housing 8. For this, the bottom section 38 of the interior housing 8 may comprise a bottom maintenance opening 54.

FIG. 1 shows the industrial water analysis device 1 in an operating position 56, in which the reactor assembly 10, in particular, the reactor tube 14 is arranged vertically. In this position, the industrial water analysis device 1 is operated.

The housing 2 comprises an access opening 60 through which the water analysis assembly 6 is accessible. The access opening 60 is usually located at a front 62 of the housing 2. A rear side 64 of the housing 2 is usually closed, e.g. by rear wall 65 of the housing 2. The access opening 60 is, during operation of the industrial water analysis device 1, usually closed, e.g. by an access door 66 or a hatch.

If the water analysis assembly 6, or, generally any component of the industrial water analysis device 1 that is located within the interior space 4, needs to be maintained, the access door 66 is opened. For opening the access door 66, the operation of the industrial water analysis device 1 does not necessarily need to be interrupted. The water analysis device 1 may even be operated when the access door 66 is open.

Access to the water analysis assembly 6, in particular to the reactor assembly 10 and/or the heating assembly 12 during maintenance may be difficult due to the limited space within the housing 2. To facilitate access, a mounting assembly 70 is provided, which is attached to the housing 2 and to the water analysis assembly 6. As shown, the mounting assembly 70 may be attached to a bottom part 72, in particular, to a bottom wall 74 of the housing 2. Between the mounting assembly 70 and the housing 2, one or more spacer 76 in the form of e.g., a bar or slat, may be provided. The at least one spacer 76 is configured to create a distance between the bottom section 38 of the interior housing 8 or the water analysis assembly 6 and the housing 2, for example, to accommodate the bottom closure 48 between the mounting assembly 70 and the housing 2.

Alternatively, the mounting assembly 70 may be mounted to a rear side 64 of the housing 2, e.g. the rear wall 65.

The mounting assembly 70 constitutes a movable mechanical connection between the water analysis assembly 6 and the housing 2, which allows to move the water analysis assembly 6 relative to the housing 2 in particular for maintenance purposes.

More specifically, the water analysis assembly 6 is attached to the mounting assembly 70 while being moveable relative to the housing 2 from the operating position 56 to a maintenance position 80.

FIG. 3 shows an example of the maintenance position 80. In the maintenance position 80, at least parts of the water analysis assembly 6 are moved out of the operating position 56 in a direction 81 pointing towards the access opening 60. More specifically, at least part of the water analysis assembly 6, preferably at least part of the top face 36 of the water analysis assembly 6 is moved closer to the access opening 60 than in the operating position 56. Preferably, at least part of the water analysis assembly 6, in particular the top section 34, protrudes through of the access opening 60 out of the housing 2 or its interior 4, respectively. It is preferred that in the maintenance position 80, the reactor cover 46 and/or the attachment means is moved out of the housing 2.

For the industrial water analysis device 1 having a maintenance opening 32 in the top section 34 and/or having a reactor cover 46, it is preferred that at least the maintenance opening 32 and/or the reactor cover 46, respectively, is located outside the interior 4 or moved through the access opening 60. The at least one inflow fluid line 22 and the at least one outflow line 51 may stay connected to the water analysis device 6 in the maintenance position 80.

In principle, the movement of the water analysis assembly from the operating position 56 to the maintenance position 80 may be purely translational, purely rotational or a combination of a rotational and a translational movement. It is only by way of example that, in FIG. 3, the water analysis assembly 6 is moved in the maintenance position 80 relative to the operating position 56 by a purely translational movement.

For such a rotational movement, the mounting assembly 70 may comprise a joint 82, in particular a hinge 84. Instead of a hinge 82, a telescopic pull-out or a parallelogram guide may be used, as used, for example, for drawers.

The hinge 82 may be arranged between a stationary part 86 of the mounting assembly 70 and a moveable part 88 of the mounting assembly 70. The joint 82 may mechanically connect the two parts 86, 88. The joint 82 may be located at a front side of the mounting assembly 70 i.e., located closer to the access openings 60 than to the rear side 64. More specifically, the joint 82 may be located at a front end 90 of the mechanical assembly 70. The hinge 82 is also preferably located at or below the bottom section 38 and/or the bottom plate 42, if present, at least in one of the operating position 56 and the maintenance position 80.

The stationary part 86 is fixed to the housing 2, while the moveable part 88 is attached to the water analysis assembly 6, in particular the interior housing 8 or the bottom face 40 or bottom plate 52 of the interior housing 8. As shown, the two parts 86, 88 may be formed as plates, manufactured e.g. from sheet metal. At least in the operating position, the water analysis assembly 6 may be fully supported by the stationary part 86.

The maintenance assembly 70 may be provided with at least one opening 91, through which the bottom closure 48 may extend. The opening 91 may be present in both the stationary part 86 and the movable part 88. In particular, the stationary part 86 and the moveable part 88 may each be provided with such an opening 91. In the operating position 56, the bottom closure 48 may extend through both the stationary part 86 and the moveable part 88. In the maintenance position 80, the bottom closure 48 may be moved away from the stationary part 86, but still extend thorugh the opening of the moveable part 86.

In the operating position, the bottom closure 48 is preferably arranged accessible through the access opening 60. For example, the bottom closure may extend below the mounting assembly 70 and the space between the bottom closure 48 and the access opening 60 may be empty.

Additional spacers may be provided between the stationary part 86 and the moveable part 88. These may adjust for the height of the joint 82. Alternatively or additionally, these spacers may cushion any impact when the water analysis assembly 6 is moved back from the maintenance position 80 to the operating position 56.

The mounting assembly 70 may further comprise a guide system 92, which in particular may be a slotted guide system that is spaced apart from the joint 82. The guide system 92 may comprise a rail or slot 94 that is configured to guide the relative movement of the water analysis assembly 6 to the housing 2 or, more specifically, the relative movement of the stationary part 86 of the mounting assembly 70 to the moveable part 88. The slot or rail 94 is mounted on one of the moveable part 88 and the stationary part 86, while a sliding member 96 engages the slot 94 or rail and is mounted on the other one of the moveable part 88 and the stationary part 86. The guide system 92 may further comprise a limit stop 98, which limits the relative movement between the stationary part 86 and the moveable part 88 to a predetermined travel length. The maintenance position 80 and the operating position 56 may be respectively located at the end of the travel length. As seen in FIGS. 1 to 3, the guide system may be arranged at the bottom section 38 of the water analysis assembly 6, in particular closer to the rear side 64 than to the front 62.

The guide system 92, together with the hinge 82, supports the water analysis assembly 6 in the maintenance position 80. In the operating position 56, the water analysis assembly 6 simply rests on the mounting assembly 70. A releasable lock 99 may be provided to secure the mounting assembly 70 and thus the water analysis assembly 6 in the maintenance position 80. The releasable lock 99 is preferably configured to be operated by hand without tools being necessary. For example, the lock 99 may comprise a wing screw that effects a frictional lock between the rail or slot 94 and the sliding member 96. Of course, any other lock, such as a catch, may be used. Preferably, the lock 99 is part of the guide system 90.

The mounting assembly 70 may further comprise at least one securing member 100 that is configured to fasten the mounting assembly 70, and thus indirectly the water analysis assembly 6, to the housing 2 in the operating position 56. The securing member 100 is preferably located spaced apart from the joint 82, and may be for example located closer to the top section 34 of the water analysis assembly 6 than to the bottom section 38.

It is preferred that the securing member 100 can be released, or detached from the housing 2, without the use of tools. For example, the securing member 100 may comprise a bayonet lock or a screw connection provided with a grip, such as a wing screw.

The mounting assembly 70 may further comprise a frame 102, into which the water analysis assembly 6 may be inserted, e.g. from the top. In the frame 102 the water analysis assembly 6 may be received. The frame 102 may be attached to the water analysis assembly 6, in particular the interior housing or interior housing 8 by fastening components 104, such as screws or clips. Again, it is preferred that the connection established between the mounting assembly 70 and the water analysis assembly 6 can be disengaged without tools, e.g. by using a wing screw.

The frame 102 may have a generally basket-like structure e.g. by comprising stanchions 106. The stanchions form insertion guides that facilitate the insertion and positioning of the water analysis assembly when mounting it to the mounting assembly 70. At least one stanchion 106 may be provided abutting a side face 108 of the interior housing 8, the side face 108 facing in a direction parallel to the plane of the access open 60. Preferably, at least one stanchion 106 is provided at each side face 108 to offer a bidirectional lateral support. At least one stanchion 106 may be provided on a front side 110 of the interior housing 8. The front side 110 faces the access opening and is located opposite a rear side 111 of the interior housing 8.

At least two stanchions 106 may have an L-shaped cross section and extend along an edge 112 of the interior housing 8, the at least one edge 112 being preferably located between the side face 108 and the front side 110. According to FIGS. 3 and 4, this may be the stanchions 106 closest to the front 62. Thus, the at least one stanchion 106 which abuts the front side 110 may support some of the weight of the water analysis assembly 6 in the maintenance position 80.

For removing the water analysis assembly 6, the water analysis assembly 6 has first to be moved from the operating position 56 in to the maintenance position 80. For this, the securing element 100 must be released.

Once the water analysis assembly 6 is in the maintenance position, the fastening elements 104 may be disengaged and the water analysis assembly 6 may be lifted as a unit out of the mounting assembly 70 and out of the housing 2. Before removing the water analysis assembly 6, the inflow or outflow fluid lines that connect the water analysis assembly 6 with the rest of the industrial water analysis device 1 should be removed.

If only the reactor assembly 10, the heating assembly 12 and/or the reactor tube 14 need be removed, then only the attachment means 44 in the top section 34 of the water analysis assembly 6 needs to be disengaged. After this, the reactor assembly 10, the heating assembly 12 and/or the reactor tube 14 may be lifted from the remainder of the water analysis assembly 6, and/or out of the interior housing 8, again once the industrial water analysis device 1 is in the maintenance position 80.

Once, maintenance is completed and the water analysis assembly 6 is connected to the at least one inflow fluid line 22 and the at least one outflow line 51, the water analysis assembly is moved back from the maintenance position 80 to the operating position 56. The at least one securing element 100 may then be engaged with the housing 2 to fix the mechanical assembly and thus the water analysis device 6 in place.

The mounting assembly 70 may be a standalone part as shown in FIG. 4, e.g. a retrofit part, which is configured to be mounted in an industrial water analysis device 1 that is not provided with such a mounting assembly 70. For this, the moveable part 88 and the stationary part 86 need only be placed between the water analysis assembly 6 and the housing 2. As the parts 86, 88 are plate-like, they do not add height to the water analysis assembly 6. The configuration of the frame 104 e.g., the placement of the stanchions 106, may be easily adapted to the geometry of the water analysis assembly 6, as is the shape of the parts 86, 88. Apart from providing, a counterpart for the securing member 100, and an attachment for the stationary plate 86 (or, in the case of e.g. a telescopic pullout) no modification to an existing industrial water analysis device 1 is needed.

### REFERENCE NUMERALS

1 industrial water analysis device
2 housing
4 interior space
6 water analysis assembly
8 interior housing/interior housing
10 reactor assembly
12 heating assembly
14 reactor tube
16 sensor assembly
18 water inflow section
20 fluid connection
22 inflow fluid line
24 water source
26 controller
27 data-transfer connection
28 inflow valve
30 memory section
32 top maintenance opening
34 top section
36 top face
37 top plate
38 bottom section
40 bottom face
42 bottom plate
44 attachment means
45 clamp or bracket
46 reactor cover
48 bottom closure
50 outflow connector
51 outflow fluid line
52 drain
53 container
54 bottom maintenance opening
56 operating position
60 access opening
62 front of housing
64 rear side of housing
65 rear wall of housing
66 access door of housing
70 mounting assembly
72 bottom part of water analysis assembly
74 bottom wall of interior housing
76 spacer
80 maintenance position
81 direction pointing from operating to maintenance position
82 joint
84 hinge
86 stationary part of mounting assembly
88 moveable part of mounting assembly
90 front end of mounting assembly
91 opening
92 guide system
94 slot or rail
96 sliding member
98 limit stop
99 lock
100 securing member
102 frame
104 fastening element
106 stanchion
108 side face of interior housing
110 front side of interior housing
111 rear side
112 edge of interior housing

1-18. (canceled)

19. Industrial water analysis device, in particular industrial TOC and/or TNb water analysis, comprising:
a housing, the housing having an access opening;
a water analysis assembly, the water analysis assembly being arranged within the housing and comprising at least one of a heating assembly and a reactor assembly, the water analysis assembly further comprising a top section and a bottom section, the top section being located above the bottom section in a vertical direction; and
a mounting assembly, the mounting assembly being attached to the housing;
wherein the water analysis assembly is attached to the mounting assembly while being movable relative to the housing from an operating position to a maintenance position;
wherein, in the maintenance position, at least parts of the water analysis assembly are moved out of the operating position in a direction pointing towards the access opening.

20. Industrial water analysis device according to claim 19,
wherein, in the maintenance position, the water analysis assembly at least partly protrudes out of the housing.

21. Industrial water analysis device according to claim 19,
wherein, in the maintenance position, at least the top section of the water analysis assembly protrudes out of the housing.

22. Industrial water analysis device according to claim 19,
wherein, in the maintenance position, the water analysis assembly is rotated relative to the operating position.

23. Industrial water analysis device according to claim 19,
wherein the mounting assembly comprises a joint.

24. Industrial water analysis device according to claim 23,
wherein the joint is located closer to the bottom section than to the top section of the water analysis assembly.

25. Industrial water analysis device according to claim 23,
wherein the water analysis assembly has a front side and a rear side, the front side facing towards the access opening and the rear side facing away from the front side; and
wherein the joint is located closer to the front side than to the rear side of the water analysis assembly.

26. Industrial water analysis device according to claim 23,
wherein a guide system is provided, the guiding system being spaced apart from the joint and comprising a guide slot or rail along which one of the water analysis assembly and the mounting assembly is guided slideably relative to the other one of the water analysis assembly and the mounting assembly.

27. Industrial water analysis device according to claim 19,
wherein the reactor assembly extends to a top section of the water analysis assembly and is arranged accessible at the top section.

28. Industrial water analysis device according to claim 19,
wherein the mounting assembly comprises a frame into which the water analysis assembly is inserted.

29. Industrial water analysis device according to claim 19,
wherein the water analysis assembly comprises an interior housing, in which the reactor assembly is received;
wherein the interior housing comprises a top maintenance opening; and
wherein the reactor assembly protrudes out of the interior housing through the top maintenance opening.

30. Industrial water analysis device according to claim 29,
wherein the reactor assembly comprises a reactor tube, the reactor tube protruding through the maintenance opening, and
wherein attachment means are provided, the attachment means being located in the top section outside the interior housing, at least one of the reactor assembly and the reactor tube being held to the interior housing by the attachment means.

31. Industrial water analysis device according to claim 29,
wherein at least one of the reactor assembly and the reactor tube are configured to be removed from the interior housing through the maintenance opening.

32. Industrial water analysis device according to claim 19,
wherein the reactor assembly comprises a reactor cover, the reactor cover being provided with at least one inflow fluid connector that is configured to be connected to a water supply;
wherein the reactor assembly is in sealing engagement with the reactor cover; and
wherein the reactor assembly is suspended from the reactor cover.

33. Industrial water analysis device according to claim 19,
wherein the mounting assembly comprises a releasable lock that is lockable at least in the maintenance position (80).

34. Industrial water analysis device according to claim 19,
wherein the water analysis assembly is connected to at least one of a water source and a fluid drain in the operating position, in the maintenance position and during movement between the operating position and the maintenance position.

35. Industrial water analysis device according to claim 19,
wherein the industrial water analysis device is configured to be connected to a water source and comprises a controller that is configured to automatically draw samples from the water source depending on a stored, predetermined schedule.

36. Mounting assembly configured to be mounted in an industrial water analysis device, the mounting assembly comprising
a stationary part configured to be attached to a housing of the industrial water analysis device;
a movable part configured to support a water analysis assembly of the industrial water analysis, the water analysis assembly comprising at least one of a heating assembly and a reactor assembly);
wherein the movable part is movable relative to the stationary part while the mounting assembly is attached to both the water analysis assembly and the housing.
